(19) United States
(12) Patent Application Publication
(10) Pub. No.: US 2023/0041289 A1
(43) Pub. Date: Feb. 9, 2023

(54) COMPOSITIONS AND METHODS FOR ENHANCING OR TREATING FEMALE SEXUAL RESPONSE

(71) Applicant: INNOVUS PHARMACEUTICALS, INC., Englewood, CO (US)

(72) Inventor: Bassam Damaj, San Diego, CA (US)

(21) Appl. No.: 17/395,326

(22) Filed: Aug. 5, 2021

(51) Int. Cl. A61K 36/30; A61K 36/23; A61K 31/522; A61K 36/53; A61K 31/375; A61K 31/355; A61K 31/192; A61K 31/366

(52) U.S. Cl. CPC A61K 36/30; A61K 36/23; A61K 31/522; A61K 36/53; A61K 31/375; A61K 31/355; A61K 31/192; A61K 31/366

(57) ABSTRACT

The present disclosure relates to compositions, articles of manufacture, methods of preparation thereof, methods of use thereof, etc., for enhancement of sexual pleasure, conditions, disorders, and diseases related to reproductive physiology and systems, especially of mammalian females.

### BACKGROUND OF THE DISCLOSURE

Sexual dysfunction is extremely common in both sexes, possibly affecting up to 40% of women (Houman et al., Current Urology Reports 2016, 17:28). There are a variety of therapies for males with sexual dysfunction, mostly focused on therapy of erectile dysfunction with drugs that inhibit phosphodiesterase 5, elevating levels of cyclic guanine monophosphate and promoting smooth muscle relaxation. This strategy has been largely ineffective for women, even when it results in clitoral engorgement. A recently released mixed serotonin receptor agonist/antagonist, which was designed to treat female sexual dysfunction, has also not proved to be effective (Jaspers et al., JAMA Internal Medicine 2016; 176:453-62).

In the years leading up to menopause, hormone levels, for example estrogen levels, can become unpredictable. Progesterone secretion also drops off and eventually stops. Androgens begin to decline. These shifts during the menopausal transition can cause issues with many aspects of a women's life, including sleep, mood, and sex.

After menopause, the loss of estrogen and androgen can lead to a drop in sexual responsiveness. This estrogen loss leads to a decrease in vaginal lubrication and vaginal elasticity, and the decline in androgens may affect interest and enjoyment. These changes, while entirely natural, can be very frustrating.

There is a need for products that are easy to use, increase intensity of sensations and level of arousal, increase orgasm ease, intensity, and frequency, increase overall satisfaction with sexual experience, and do not produce an unappealing smell.

### SUMMARY OF THE DISCLOSURE

In some embodiments, the disclosure relates to methods of improving a sexual characteristic, comprising administering a composition comprising borage seed oil, evening primrose oil, Angelica pubescens root powder, and theobromine. In some aspects the composition comprises about 47% borage seed oil w/w, about 47% evening primrose oil w/w, and about 64% Angelica pubescens root powder w/w. In some aspects, the composition comprises about 0.46% theobromine. In some aspects the composition further comprises an ingredient selected from the group consisting of Coleus forskohlii extract, ascorbyl palmitate, alpha tocopherol, ferulic acid, α-Angelica lactone, and osthole. In some the composition further comprises Coleus forskohlii extract, ascorbyl palmitate, alpha tocopherol, ferulic acid, α-Angelica lactone, and osthole.

In some embodiments, the composition further comprises an ingredient selected from the group consisting of Coleus forskohlii extract at a final concentration of about 0.46% w/w, ascorbyl palmitate at a final concentration of about 0.46% w/w, alpha tocopherol at a final concentration of about 0.38% w/w, ferulic acid at a final concentration of about 0.46% w/w, α-Angelica lactone at a final concentration of about 0.48% w/w, and osthole at a final concentration of about 0.94% w/w. In some aspects, the composition further comprises Coleus forskohlii extract at a final concentration of about 0.46% w/w, ascorbyl palmitate at a final concentration of about 0.46% w/w, alpha tocopherol at a final concentration of about 0.38% w/w, ferulic acid at a final concentration of about 0.46% w/w, α-Angelica lactone at a final concentration of about 0.48% w/w, and osthole at a final concentration of about 0.94% w/w. In some embodiments, the composition further comprises flavoring. In some aspects, the flavoring is selected from the group consisting of raspberry flavoring, peach flavoring, neotame, ammoniated glycyrrhizin, and monoammonium glycyrrhizinate.

In some embodiments, the methods increase intensity of sensations, increase level of arousal, increase orgasm ease, increase intensity, increase frequency, increase overall satisfaction with sexual experience.

In some embodiments, the composition does not produce an unappealing odor.

In some embodiments the disclosure relates to compositions comprising an oil base of borage seed oil, evening primrose oil, and Angelica pubescens, wherein the oil base is produced by a process comprising mixing borage seed oil with evening primrose oil, mixing Angelica pubescens root with the borage seed oil and evening primrose oil, clarifying the resulting mixture, adding Fuller's Earth to the mixture and mixing further to produce a second mixture, and clarifying the second mixture.

In some embodiments, the composition does not produce an unappealing odor.

In some embodiments, the composition comprises an additional ingredient selected from the group consisting of Coleus forskohlii extract, ascorbyl palmitate, alpha tocopherol, ferulic acid, α-Angelica lactone, theobromine, and osthole. In some aspects, the additional ingredient is added to the oil base.

In some aspects, the composition does not produce an unappealing odor.

In some embodiments, the composition comprises the additional ingredients Coleus forskohlii extract, ascorbyl palmitate, alpha tocopherol, ferulic acid, α-Angelica lactone, theobromine, and osthole.

In some embodiments, the additional ingredients are added to the oil base.

In some embodiments, the composition does not produce an unappealing odor.

In some embodiments, the composition comprises about 47% borage seed oil w/w, about 47% evening primrose oil w/w, about 64% Angelica pubescens root powder w/w, about 0.46% Coleus forskohlii extract w/w, about 0.46% ascorbyl palmitate w/w, about 0.38% alpha tocopherol w/w, about 0.46% ferulic acid 0.46% w/w, about 0.48% α-Angelica lactone w/w, and about 0.94% osthole w/w.

### BRIEF DESCRIPTION OF THE FIGURES

FIG. 1 shows the results of consumer survey questions regarding ease of use of a composition provided by the present disclosure (ZESTRA®) and its dosing instructions.

FIG. 2 shows the results of consumer survey questions on the intensity of sensations and level of arousal produced when using a composition provided by the present disclosure (ZESTRA®).

FIG. 3 shows the results of consumer survey questions on the ease of achieving orgasm, orgasm intensity, and orgasm frequency when using a composition provided by the present disclosure (ZESTRA®).

FIG. 4 shows the results of a consumer survey question on the overall satisfaction with sexual experience before and when using a composition provided by the present disclosure (ZESTRA®).

FIG. 5 shows the results of consumer survey questions on frequency of use, likelihood of continuing use, and likelihood of recommending to others of a composition provided by the present disclosure (ZESTRA®).

FIG. 6 shows the results of a consumer survey question on the appeal of the smell of a composition provided by the present disclosure (ZESTRA®).

### DETAILED DESCRIPTION

The present disclosure relates to all aspects of modulating the female sexual response, including female sexual dysfunction, such as female sexual arousal disorders (FSAD), orgasmic disorders, and sexual pain disorders, and to enhancing female sexual pleasure and satisfaction of the female sexual experience. In various aspects, the present disclosure relates to compositions and/or articles of manufacture, as well as to their methods of preparation and methods of use, for conditions, disorders, and diseases related to female reproductive physiology systems, inclusive of those involved in the female sexual response. Accordingly, the present disclosure relates to compositions comprising botanical extracts, active agents, and related components, which are useful to treat or affect any of the aforementioned conditions. For example, in some embodiments the present disclosure provides a composition for topical or local use, which comprises one or more of the following ingredients: borage seed oil, evening primrose oil, Angelica pubescens root (and/or the root of other Angelica species), Coleus forskohlii extract, α-Angelica lactone, theobromine, osthole, ferulic acid, theobromine, ascorbyl palmitate, vitamin E and equivalents thereof, as well as any and all combinations of the foregoing.

Compositions provided by the present disclosure can be produced and used in accordance with the present disclosure such that they are useful to treat or affect the female sexual response. For example, in some embodiments the present disclosure provides compositions for topical or local use, which comprise one or more of the following ingredients: borage seed oil and other sources of gamma linolenic acid (GLA), Angelica pubescens root, Coleus forskohlii extract, and other naturally-occurring cyclic adenosine monophosphate (cAMP) and cyclic guanine monophosphate (cGMP) phosphodiesterase (PDE) inhibitors and equivalents thereof, as well as any and all combinations of the foregoing. The compositions are effective to produce one or more of the following effects: increases in localized nitric oxide, cAMP production and/or elevation, cGMP production and/or elevation, prostaglandin E₁ production, inhibition of prostaglandin E₁ breakdown, calcium channel antagonism, phosphodiesterase inhibition, anti-oxidation, vasodilation, smooth muscle relaxation, and/or combinations thereof.

In some embodiments, the present disclosure provides compositions comprising borage seed oil or other borage plant parts, for example from Borago officianalis. The borage plant (e.g., leaves, roots, and seeds) comprises a complex mixture of defined and undefined constituents, including, acetic acid; alkaloids; allantoin; amabiline; arabinose; ascorbic-acid; beta-carotene; bornesitol; calcium; choline; cobalt; dhurrin; fat; fiber; galactose; gamma linolenic acid; glucose plant; intermedine; lycopsamine; magnesium; malic acid; mucilage; niacin; phosphorus potassium; protein; pyrrolizidines; resin; riboflavin; rosmarinic acid; silicic acid; sodium; supinine; supinine viridiflorate; thiamin and zinc. Borage seed oil also contains gamma linolenic acid (GLA) having a molecular weight of 278. GLA is a polyunsaturated fatty acid (PUFA) belonging to the group of fatty acids called omega-6 or N-6 fatty acids because of the presence of a double bond between the 6th and 7th carbon. GLA is found in the seed of the Borage plant and is also found in evening primrose seed oil and other botanical and natural sources. Borage see oil can contain 11%, 21%, and/or 20-26% GLA content. Evening primrose oil can contain 9%, 11%, 21%, and/or 20-26% GLA content.

Borage seed oil can be prepared by any suitable method, for example methods which extract GLA and other bioactive agents all at once, such as cold pressure extraction, screw pressure extraction, solvent extraction, and supercritical fluid extraction. Borage seed oil can be extracted and/or otherwise formulated to comprise any amount of GLA by weight. In various embodiments. Compositions provided by the present disclosure can comprise borage seed oil in an amount selected from about 10%, about 15%, about 20%, about 25%, about 30%, about 35%, about 40%, about 45% and about 50%. In such embodiments the borage seed oil preferably is free of compounds which are toxic, or deleterious to mammals, such as alkaloids and pyrrolizidine.

In some embodiments, borage seed oil can be present in a composition of the present disclosure in an effective amount selected from about 1% to about 50%, about 5% to about 45%, about 10% to about 40%, about 20% to about 30%, about 20% to about 25%, about 21%, about 22%, about 23%, about 24% or about 25%, w/w (weight of ingredient/weight of total composition).

In addition to borage seed oil, other sources of GLA can be utilized in the disclosed compositions, including, for example, purified or isolated GLA, botanical extracts such as evening primrose oil (e.g., Oenothera biennis and Oenothera lamarckiana), black currant oil, spirulina, oils from the seeds of the Ribes family, and others.

Borage seed oil has a variety of beneficial effects and activities, including, but not limited to, inhibiting platelet aggregation, lowering blood pressure, anti-inflammatory activity, vasodilation, prostaglandin promoting activity, PGE₁ promoting activity, promoting circulating hormones, causing smooth muscle relaxation, and others. in various aspects, compositions provided by the present disclosure comprise borage seed oil in amounts which are effective to achieve one or more of the aforementioned effects.

The evening primrose (Oenothera biennis; plant family Onagraceae) is a plant that opens its bright yellow blossoms when the weather is dull or in the evening, hence the name evening primrose. The oil obtained from the seeds of the evening primrose (Oleum Oenotherae) contains a high concentration of polyunsaturated fatty acids, such a linoleic acid and gamma linolenic acid.

Compositions provided by the present disclosure comprise evening primrose oil. Evening primrose oil can be present in a composition of the present disclosure in any effective amount, for example from about 1% to about 50%, about 5% to about 45%, about 10% to about 40%, about 20% to about 30%, about 20% to about 25%, about 21%, about 22%, about 23%, about 24% or about 25%, w/w (weight of ingredient/weight of total composition).

Compositions provided by the present disclosure can also comprise Angelica, such as Angelica archangelica, Angelica sinensis, Angelica sylvestris, Angelica officinalis, archangel, European angelica, garden angelica, Angelica acutiloba, and/or Angelica pubescens which is also known as Du Huo or Du Huo Radix. In some embodiments the composition comprises Angelica root, but other parts of the plants can be used as well. Angelica contains a wide and complex variety of different constituents, such as flavonoids, flavones and coumarins, preferably, osthole or 7-methoxy-8-(3-methylpent-2-enyl)coumarin, alpha-angelicalactone, other coumarins including meranzin hydrate, nodakentin, marmesinin, columbianadin, columbianetin, bergapten, heramandiol, 6-alkylcoumarins, angelol-type coumarins, byak-angelicin, ferulin, oxypeucedanin, umbelliprenin, imperatorin, neobyakangelicin, prenylcourmarins, glabralactone, anpubesol, angelical, angelin, furanocourmins, and derivatives thereof. Other bioactive agents include linoleic acid, osthenol, falcarindiol, numerous flavinoids and flavones, 11(S), 16(R)-dihydroxyoctadeca-9Z,17-diene-12,14-diyn-1-yl-acetate, xanthotoxin, umbelliferone, ferulic acid, magnesium, and derivatives thereof.

Angelica is useful to elicit a number of pharmacological activities, including smooth muscle relaxant activity, phosphodiesterase inhibition, calcium antagonist activity, cycloxygenase and 5-lipoxygenase inhibition (Liu et al., Pharm. Bio., 36(3):207-216, 1998). The coumarins present in Angelica, osthole in particular, have been shown to display activities such as inhibition of platelet aggregation, inhibition of smooth muscle contraction, smooth muscle relaxation (Che-Ming et al., Naunyn-Schmiedeberg's Arch. Pharmacol., 349:202-208, 1994), inhibition of calcium flux, cyclic nucleotide (such as cGMP and cAMP) phosphodiesterase inhibition, increase in cAMP and cGMP levels, anti-proliferative activity, anti-inflammatory activity (Yuh-Fung et al., Planta Medica, 61(1):2-8, 1995), enhancement of the increase of cAMP and cGMP induced by forskolin, vasorelaxation, and to aid in neurotransmitter receptor binding such as GABA, 5HT-1A, D-2, and D-1 receptors (Jyh-Fei et al., Proceedings of the National Science Council Republic of China, Part B, Life Sci., 19(3):151-158, 1995). Alpha-angelicalactone is useful to elicit various activities, including calcium antagonism (Entman et al., J. Clin. Invest., 48:229-234, 1969). Ferulic acid, another component of Angelica root, has been shown to scavenge oxygen free radicals and increase intracellular cAMP and cGMP (Zheng R L, Zhang H., Free Radic Biol Med., 22(4):581-586, 1997). In various aspects, compositions provided by the present disclosure are formulated to take advantage of Angelica activities such as cyclic nucleotide phosphodiesterase inhibition, calcium antagonism, oxygen free radical scavenging, smooth muscle modulation, vasorelaxation and/or vasodilation.

Compositions provided by the present disclosure can comprise any effective amount of Angelica and/or Angelic pubescens root, for example from about 10% to about 50%, about 20% to about 40%, about 25% to about 35%, about 26%, about 27%, about 28%, about 29%, about 30%, about 31%, about 32%, about 33%, or about 34% w/w.

Compositions provided by the present disclosure can further comprise Coleus forskohlii, in some embodiments from its tuber or roots. The plant is a member of the Labiatae family and grows as a perennial. It is native to India, Nepal, Sri Lanka, and Thailand (The Wealth of India, Vol. II, C.S.I.R., India, 1950, Page 308). Coleus forskohlii comprises a diverse and complex mixture of compounds including diterpenes and derivatives thereof, for example forskolin and related diterpenes. Coleus forskohlii can contain about 80% forskolin.

Extracts of coleus forskohlii can be prepared routinely by contacting the plant parts with a suitable solvent to extract a diterpene or other compound from the material (U.S. Pat. No. 4,118,508, JP 11292777, and JP 6133731 for extraction processes). Compositions provided by the present disclosure can comprise any effective amount of Coleus, for example from about 0.1 to about 10%, about 1% to about 9%, about 2% to about 8%, about 3%, about 4%, about 4.5%, about 4.6%, about 5%, about 6%, about 7% w/w.

Coleus forskohlii, particularly forskolin and related diterpenes, can elicit a number of biological activities, including, smooth muscle relaxation, adenylate cyclase stimulation, elevation of levels of cAMP, anti-inflammatory, anti-spasmodic, and others. Since forskolin and related diterpenes stimulate adenylate cyclase, resulting in the production of the second messenger cAMP, any biological process mediated by cAMP can therefore be stimulated by administration of Coleus forskohhi.

In various aspects, compositions provided by the present disclosure also comprise ferulic acid. Any amount of ferulic acid which is effective can be utilized in compositions of the present disclosure, for example from about 0.1% to about 10%, about 1% to about 9%, about 2% to about 8%, about 3%, about 4%, about 4.5%, about 4.6%, about 5%, about 6%, or about 7% w/w.

Theobromine (C₇H₈N₄O₂, chemical name 3,7-dimethylxanthine or 3,7-dihydro-3,7-dimethyl-1H-purine-2,6-dione) is an alkaloid from the methylxanthine family, a family that also includes theophylline and caffeine. Theobromine has two methyl groups in comparison with the three groups that caffeine contains.

In various aspects, compositions provided by the present disclosure also comprise theobromine. Any amount of theobromine which is effective can be utilized in compositions of the present disclosure, for example from about 0.1% to about 10%, about 1% to about 9%, about 2% to about 8%, about 3%, about 4%, about 4.5%, about 4.6%, about 5%, about 6%, or about 7% w/w.

In various aspects, compositions provided by the present disclosure also comprise α-Angelica lactone. Any amount of α-Angelica lactone which is effective can be utilized in compositions of the present disclosure, for example from about 0.1% to about 10%, about 1% to about 9%, about 2% to about 8%, about 3%, about 4%, about 4.7%, about 4.8%, about 5%, about 6%, or about 7% w/w.

In various aspects, compositions provided by the present disclosure also comprise osthole. Any amount of osthole which is effective can be utilized in compositions of the present disclosure, for example from about 1% to about 20%, about 5% to about 15%, about 6% to about 14%, about 7%, about 8%, about 9%, about 9.3%, about 9.4%, about 9.5%, about 10%, about 11%, about 12%, about 13%, or about 14% w/w.

In various aspects, compositions provided by the present disclosure also comprise ascorbyl palmitate. Any amount of ascorbyl palmitate which is effective can be utilized in compositions of the present disclosure, for example from about 0.1% to about 10%, about 1% to about 9%, about 2% to about 8%, about 3%, about 4%, about 4.5%, about 4.6%, about 5%, about 6%, or about 7% w/w.

In various aspects, compositions provided by the present disclosure also comprise alpha tocopherol, DL-alpha tocopherol, and/or other form of vitamin E. Any amount of alpha tocopherol, DL-alpha tocopherol, and/or other form of vitamin E which is effective can be utilized in compositions of the present disclosure, for example from about 0.1% to about 10%, about 1% to about 9%, about 2% to about 8%, about 3%, about 3.8%, about 3.9%, about 4%, about 5%, about 6%, or about 7% w/w.

In various aspects, compositions provided by the present disclosure also comprise flavoring. Any amount of flavoring which is effective can be utilized in compositions of the present disclosure, for example from about 0.1% to about 2%, about 0.2% to about 1.8%, about 0.5% to about 1.5%, about 0.8%, about 0.9%, about 1%, about 1.1%, about 1.2%, about 1.3%, or about 1.4% w/w. Suitable flavorings can include, for example raspberry, peach, mango, other fruit flavoring, other palatable flavoring, or any combination thereof.

In various aspects, compositions provided by the present disclosure also comprise a sweetener. Any amount of sweetener which is effective can be utilized in compositions of the present disclosure, for example from about 0.05% to about 0.5%, about 0.1% to about 4%, about 0.2% to about 0.3%, about 0.21%, about 0.22%, about 0.23%, about 0.24%, about 0.25%, about 0.26%, about 0.27%, about 0.28%, about 0.29% w/w, about 0.1% to about 1%, about 0.2% to about 0.9%, about 0.3% to about 0.8%, about 0.4 to about 0.7%, about 0.45%, about 0.5%, about 0.55%, or about 0.6%. Suitable sweeteners can include, for example neotame, ammoniated glycyrrhizin, monoammonium glycyrrhizinate, or other forms of glycyrrhizic acid, including salt forms such as ammonium salt forms, or any combination thereof.

In addition to the above-mentioned botanical extracts, or as an alternative thereof, a composition of the present disclosure can comprise any agent which possesses one of more of the biological activities associated with said botanical extracts.

For example, the present disclosure also provides compositions comprising agents having “PGE₁ promoting activity.” As mentioned, one of the major constituents of borage seed oil is GLA. GLA is a precursor of prostaglandin E1 (PGE₁), a potent biological effector molecule capable of eliciting many physiological effects. While not wishing to be bound by any theory, it is believed that at least some of the beneficial effects produced by borage seed oil is mediated by its delivery of a precursor to the PGE₁ metabolic pathway, thereby stimulating production of PGE₁. Thus, any compound, mixtures thereof, compositions, and/or botanicals which comprise a PGE₁ or a PGE₁ precursor can be characterized as having “PGE₁ promoting activity” and/or as causing the production of PGE₁, or possessing PGE₁ activity.

Another useful class of agents that may be included into compositions provided by the present disclosure are those which elevate levels of cyclic nucleotides, such as cAMP and cGMP, by inhibiting phosphodiesterases which hydrolyze the cyclic nucleotides, by stimulating adenylate cyclase, or receptors coupled thereto, by acting on G-proteins. In accordance with the present disclosure, the disclosed compositions can be formulated to elicit any amount of elevation or increase of cyclic nucleotide which is effective to elicit a desired result, such as treating FSD, enhancing sexual arousal, and the like. Amounts of increase as compared to normal can be at least 5%, 10%, 50%, 75%, 90%, 1-fold, 2-fold, 5-fold, 10-fold, 20-fold. These increases can be sudden, transient over a few minutes, and/or localized, as long as the desired effect is achieved, for example modulating the female sexual response.

Elevation of levels of cyclic nucleotides can be accomplished by cyclic nucleotide phosphodiesterase (PDE) inhibition. There are a number of different cyclic nucleotide phosphodiesterase isoenzymes, including types I, II, III, IV, V, VI, and VII (Nicolson et al., 1991). PDE inhibitors can be non-selective (e.g., theophylline or caffeine), or selective for one or more PDE isoenzymes. Selective inhibitors, include, I (vinpocetine), III (milrinone, amrinone, pimobendan, cilostamide, enoximone, peroximone, vesnarinone), IV (rolipram, R02-1724), and V (zaprinast, dipyridamole). Other useful PDE inhibitors include compounds disclosed in U.S. Pat. No. 5,958,926.

In addition to elevating levels of cAMP through inhibition of PDEs (e.g., utilizing Angelica pubsescens root extract and other species of Angelica) cAMP levels can be elevated by directly stimulating adenyate cyclase and causing synthesis of cAMP, e.g., using Coleus forskohlii, and derivatives thereof. Useful forskolin derivatives, and their synthesis, are disclosed in, e.g., EP 222,413, U.S. Pat. Nos. 5,789,439, 5,350,864, 5,206,241, 5,177,207, 5,145,855, 5,093,336, 4,999,351, and 4,134,986.

Compositions can be administered in any form by any effective route, including, e.g., oral, parenteral, enteral, intraperitoneal, topical, local, dermal, transdermal, ophthalmic, nasally, nasopharyngeal absorption, local, topical, non-oral, aerosal, inhalation, subcutaneous, intramuscular, buccal, sublingual, rectal, vaginal, intra-arterial, rapid infusion, intravenously, long-release implants, etc.

In certain embodiments of the disclosure, compositions are administered to the external female genitalia and/or vaginally, e.g., as a vaginal cream, foam, gel, jelly, liquid, oil, emulsion, solution, suspension, cream, spray, powder, suppository, tablet, device, etc. For example, a composition can be preferably applied to the female external genitalia, such as the mons pubis, labia majora and minora, hymen, clitoris, prepuce of the clitoris, vestibule of the vagina, and/or vestibular glands. The external genitalia is also called the vulva or pudendum. Compositions can also be applied to the internal wall of the vagina, e.g., to the adventia, muscularis, mucosa, and rugae.

A composition of the present disclosure can also be administered by or in the form of a device, such as a cartridge, diaphragm, female mechanical barrier-type device, feminine cap (e.g., U.S. Pat. Nos. 4,858,624, 4,989,618, and 5,207,232), film, intrauterine barrier-type device, sponge, tampon, osmotic drug delivery device (e.g., U.S. Pat. Nos. 5,795,591, 4,475,916, and 4,093,708), ring, or sheath. Such devices can carry the composition in any effective manner, e.g., a device can be impregnated or coated with the composition, or fitted with a carrier element, such as a film (e.g., U.S. Pat. No. 5,529,782), or polymeric material, etc., that contains composition. The device can then be inserted into the vagina where delivery is effected. See, e.g., U.S. Pat. No. 4,317,447. A device can be a sponge-like structure, such as a polymeric sponge tampon, which contains a composition of the present disclosure. See, e.g., U.S. Pat. No. 4,393,871. Such a device can be inserted into the vagina prior to intercourse. The device can also be reusable. In the case of devices, it can be advantageous to formulate the composition with compounds, such as water-soluble polymers or dissolvable materials, which disintegrate in the vaginal fluids, thereby releasing the active agents. Any suitable polymer can be used, e.g., as described in U.S. Pat. Nos. 5,840,685 5,393,528, 5,084,277, 4,835,138, 4,323,548, and 4,322,399.

A composition of the present disclosure can also be administered by a male condom, e.g., by applying the composition to the condom prior to insertion into the vagina, e.g., in combination with other lubricants, or, as a dry or wet film or coating on the exterior of the condom surface. See, e.g., U.S. Pat. No. 5,954,054.

In general, any delivery means, including devices, polymers, etc., that are used to deliver agents vaginally can be utilized in accordance with the present disclosure, such as means for delivering antiviral agents, bacteriocides, contraceptives, hormones, spermicides, virucides, lubricants, etc.

Compositions of the present disclosure can further comprise other active agents, including, e.g., contraceptive agents, spermicidal agents, such as, e.g., nonoxynol-9, octoxynol, menfegol, benzalkonium chloride, peroxygen compounds or hydrogen peroxide (e.g., U.S. Pat. No. 5,778,886), bacteriocides, germicides, antiviral agents, virucides, vasodilators, agents which increase vaginal lubrication (e.g., hydriodic acid syrup as disclosed in U.S. Pat. No. 5,470,588), etc.

In addition, compositions of the present disclosure can further comprise any agent which enhances the sexual response and/or treat diseases and conditions related to sexual dysfunction. Such agents include, e.g., apomorphine (e.g., U.S. Pat. No. 5,945,117), nitric oxide releasing compounds (e.g., U.S. Pat. No. 5,877,216), ginkgo (e.g., U.S. Pat. No. 5,897,864), hydriodic acid (U.S. Pat. No. 5,470,588), agents disclosed in U.S. Pat. No. 4,521,421.

The compositions of the present disclosure can further comprise any pharmaceutically acceptable carrier. By the phrase, “pharmaceutically acceptable carrier,” it is meant any excipient, solvent, vehicle, inert ingredient, etc., which is formulated with the active ingredients of a pharmaceutical composition, such as the standard agents described, e.g., in Remington's Pharmaceutical Sciences, Eighteenth Edition, Mack Publishing Company, 1990. Examples of suitable carriers are well known in the art and can include, but are not limited to, water, phosphate buffered saline solutions, phosphate buffered saline containing Polysorbate 80, emulsions such as oil/water emulsion and various type of wetting agents, salt solutions, alcohols, gum arabic, vegetable oils, benzyl alcohols, aqueous vehicles, water-miscible vehicles, nonaqueous vehicles (e.g., corn oil, cottonseed oil, peanut oil, sesame oil, ethyl oleate, isopropyl myristate, and benzyl benzoate), etc. Carriers also include, e.g., milk, sugar, certain types of clay, silica, gelatin, stearic acid or salts thereof, magnesium, magnesium stearate and other forms or salts of magnesium, or calcium stearate, talc, vegetable fats or oils, gums, glycols, propylene glycol, buffers, antimicrobial agents, preservatives, flavor, fragrance and color additives, gelatin, carbohydrates such as lactose, amylose or starch, talc, silicic acid, viscous paraffin, perfume oil, fatty acid monoglycerides and diglycerides, pentaerythritol fatty acid esters, hydroxy methylcellulose and the like. Other additives include, e.g., antioxidants and preservatives, coloring, flavoring and diluting agents, emulsifying and suspending agents, such as acacia, agar, alginic acid, sodium alginate, bentonite, carbomer, carrageenan, carboxymethylcellulose, cellulose, cholesterol, fatty acids, triglycerides and esters of fatty acids, fatty alcohols, gelatin, hydroxyethyl cellulose, hydroxypropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, transdermal enhancers (ethanol, propylene glycol, water, sodium oleate, leucinic acid, oleic acid, capric acid, sodium caprate, capric/caprylic triglyceride, silica, lauric acid, sodium laurate, neodecanoic acid, dodecyl-amine, cetryl lactate, myristyl lactate, lauryl lactate, methyl laurate, phenyl ethanol, hexa-methylene lauramide, urea and derivatives, dodecyl N, N-dimethylamino acetate, hydroxyethyl lactamide, phyophatidylcholine, sefsol-318 (a medium chain glyceride), isopropyl myristate, isopropyl palmitate, palmitic acid, several surfactants, including poly-oxyethylene (10) lauryl ether (Brij 361 R), diethyleneglycol lauryl ether (PEG-2-L), laurocapram (Azone; 1,1-dodecylazacycloheptan-2-one), acetonitrile, 1-decanol, 2-pyrrolidone, N-methylpyrrolidone, N-ethyl-1-pyrrolidone, 1-Methyl-2-pyrrolidone, 1-lauryl-2-pyrrolidone, sucrose monooleate, dimethylsulfoxide (DMSO) about 80% concentration required, decylmethylsulfoxide (n) enhances primarily polar or ionic molecules (soluble in ethanol), acetone, polyethylene glycol 100-400 MW, dimethylacetamide, dimethylformamide, dimethylisosorbide, sodium bicarbonate, various N7-16-alkanes, mentane, menthone, menthol, terpinene, D-terpinene, dipen-tene, N-nonalool and limonene, skin penetration enhancers (e.g., lecithin), and miscellaneous ingredients such as microcrystalline cellulose, citric acid, dextrin, dextrose, liquid glucose, lactic acid, lactose, magnesium chloride, potassium metaphosphate, starch, and the like.

As mentioned, compositions of the present disclosure can comprise one or more of the following ingredients, e.g., borage seed oil, Angelica pubescens root, Coleus forskohlii extract, ferulic acid, and equivalents thereof, in any binary, trinary, etc., combination. Examples of topical compositions, include, e.g., binary combinations, such as an effective amounts of borage seed oil, and Angelica pubescens root; effective amounts of borage seed oil, and Coleus forskohlii extract; effective amounts of Angelica pubescens root, and Coleus forskohlii extract; and quaternary combinations, such as effective amounts of borage seed oil, Angelica pubescens root, Coleus forskohlii extract. Such compositions can further comprise pharmaceutically-acceptable excipients, skin- and mucosal penetration enhancers, etc. In preferred embodiments, included as excipients are, e.g., de-ionized water (e.g, about 0.5-50%, for example 5%, concentration), Span 80 (sorbitan monooleate (e.g., 0.2-20%, preferably 2%, concentration), lecithin (e.g., egg or soy phosphatidylcholine (e.g., e.g., 0.2-20%, preferably 2%, concentration), lavendar for body oils by Flavor and Fragrance Specialties (0.05-1.25%, preferably 0.25%), Blackberry Musk for body oils by Flavor and Fragrance Specialties (0.1-2.5%, preferably 0.5%) or other flavors and fragrances, glycerin (e.g., 2-10% w/w), saccharin or other sweetening agents, and monosodium guanosine mono phosphate (flavor enhancer), silica, ferulic acid and other forms of ferulate, vitamine E acetate and other forms of tocopherol, and ascorbyl palmitate and other forms of ascorbic acid along with other anti-oxidants and stability enhancers. Ingredients, and amounts of ingredients, can be adjusted such that the compositions possess minimal irritation to the female reproductive organs. Ingredients can also be included that enhance the cosmetic appeal (e.g., enhancing the smell, feel, etc.) of the compositions, but which are inert as far as enhancing the sexual response, e.g., enhancing the smell, feel, etc., of a composition.

The present disclosure also relates to methods of using any of the mentioned compositions, e.g., for treating or affecting diseases and conditions associated with sexual function, especially associated with the female reproductive system, such as for treating sexual dysfunction, facilitating sexual arousal, enhancing or improving sexual response, or enhancing or improving sexual pleasure, comprising administering an effective amount of a composition in accordance with the present disclosure. By “sexual functioning,” it is meant any activity associated with the genitalia, such as sexual intercourse. The methods are useful to treat various types of female sexual dysfunction (FSD), such as female sexual arousal disorder (FSAD), desire disorders, orgasmic disorders, and sexual pain disorders. Premenopausal and post-menopausal women can be treated.

The stages of female sexual activity include excitement (arousal), plateau, and orgasm. The arousal response is a physiological and psychological process involving, e.g., muscle relaxation, vasocongestion, vasodilation, and muscular contraction. The clitoris which contains a rich supply of sensory endings becomes erect as a result of vasocongestion. During intercourse, the vaginal epithelium becomes highly congested and secretes a mucus-like lubricant which is an exudate. See, e.g., Human Physiology, Vander et al., Fifth Edition, McGraw-hill Publishing Company, 1990, e.g., Page 628; Current Medical Diagnosis, Tierney et al., Eds., 1997, e.g., Pages 962-965; U.S. Pat. No. 5,958,926, especially Column 7-9. Compositions of the present disclosure can particularly facilitate and/or enhance arousal and orgasm, e.g., by enhancing associated vasocongestion and vasodilation and sensory input.

Sexual arousal disorders, e.g., inability to become aroused or inability to attain or maintain sufficient sexual excitement, female impotence, vaginismus, frigidity, disorders of sexual desire, e.g., absence of libido, decreased or loss of sensation, etc. can be treated or affected in accordance with the present disclosure. In addition, sexual pain disorders, such as painful intercourse, or dyspareunia, can be treated. The latter can be caused by a number of factors, including, e.g., endometriosis, vaginismus, insufficient lubrication of the vagina, etc. F SAD can be manifested by a patient as a lack of subjective excitement, a lack of genital lubrication or swelling, or another somatic responses. Disorders of arousal include, but are not limited to, lack or diminished vaginal lubrication, decreased clitoral and labial sensation, decreased clitoral and labial engorgement, lack of vaginal smooth muscle relaxation, and disorders involving hormonal status. Compositions of the present disclosure can treat any of the mentioned conditions associated with female sexual dysfunction.

In addition, the compositions are useful for enhancing or improving sexual response and/or enhancing or improving sexual pleasure and sensation. By the terms, “enhance” or “improve,” it is generally meant that administration of a composition increases the subject's satisfaction with the sexual activity as compared to the activity when in the absence of the composition. This includes, e.g., enhancement of vaginal wetness, warmth, engorgement, sensitivity, sensation, tingling, arousal, orgasm, quicker to arousal, quicker to orgasm, and enhancement of any of the above-mentioned conditions (e.g., clitoral and labial sensation or vaginal smooth muscle relaxation), etc. Any amount of increase in satisfaction can be achieved, including, e.g., 1%, 5%, 10%, 50%, 100%, 2-fold, etc. Satisfaction can be determined by any suitable method, e.g., a survey or questionnaire in which a user is asked to assess, after using a composition of the present disclosure, changes in the genital area and sexual pleasure.

By the term “administering,” it is meant that a composition is delivered to the subject in such a way that it can achieve the desired purpose, e.g., treating a condition or disease associated with sexual function. As mentioned, such composition can be administered by any effective route, preferably vaginally, such as topically or locally. Compositions of the present disclosure can be administered to any suitable subject, preferably human females, but also to females of other species, such as apes, monkeys, chimpanzees, pets, such as dogs, cats, rats, hamsters, and mice, horses, pigs, cows, sheep, and other domestic animals, and males of any species.

In addition to females having any of the aforementioned conditions, suitable female subjects, include, e.g., females having illnesses that interfere with sexual arousal, such as diabetes mellitus, hypothyroidism, pelvic disorders, neurological disorders (e.g., multiple sclerosis), muscular disorders (e.g., muscular dystrophy), and psychological disorders (guilt, anxiety, depression, fatigue, or interpersonal conflicts), conditions that lead to failure of the vasocongestion response, vaginal dryness, anorgasmic females, intermittently orgasmic females, orgasmic females desiring greater sexual response, sexual-related failures associated with age, neurosis, females having sexual desire disorders, orgasmic dysfunction, drug-induced sexual dysfunction (e.g., associated with oral contraceptives, anti-hypertensives, tranquilizers, SSRI antidepressants), hypoactive sexual desire (HSD), postmenopausal women, etc.

Administration of compositions of the present disclosure can alleviate, improve, or ameliorate any of the mentioned conditions. In addition, sexual response can be improved, e.g., decreasing foreplay (e.g., the time usually required for a subject to reach arousal), decreasing latency time between orgasms, decreasing the time to reach orgasm, and facilitating orgasm and multiple orgasm.

The disclosed methods and compositions are useful to increase intensity of sensations and level of arousal, increase orgasm ease, intensity, and frequency, increase overall satisfaction with sexual experience, and do not produce an unappealing smell.

An effective amount of a composition is administered to a target subject. Effective amounts are such amounts that are useful to achieve the desired effect, preferably a beneficial, pleasurable or therapeutic effect as described above. Such amounts can be determined routinely, e.g., by administering different dosages to subjects and surveying or questioning such subjects after sexual activity about their preferences and the effectiveness of the treatment. Amounts can be selected based on various factors, including the age, health, gender, and weight of the subject.

### EXAMPLES

### Example 1. Preparation of ZESTRA Oil Base

Borage seed oil (e.g., containing 11%, 21%, and/or 20-26% GLA content) was mixed with evening primrose oil (e.g., 9%, 11%, 21%, and/or 20-26% GLA content) at a 1:1 weight/weight ratio. Milled Angelica pubescens root was mixed in to the borage seed oil/evening primrose oil at 63.897% w/w ratio of Angelica pubescens root to oil for 48-72 hours at 1750 mixer revolutions per minute (rpms). The mixture was warmed to 37 degrees C. and then passed through a peristaltic pump and centrifuged, prior to filtration of the liquid through a 10 micron filter and then a 2 micron filter. The clarified mixture was then combined with room temperature water, at 46.64% mixture and 53.34% water.

The resulting mixture was then agitated for 45 minutes at a mixing speed of 1250 rpms, cooled to 9-12 degrees C. and allowed to sit for 12 hours. The mixture was then heated to 37 degrees C. and passed through a peristaltic pump and centrifuged. The resulting liquid fraction was then combined with Fuller's Earth (a clay material for decolorization and purification of oil and/or other liquids) at 18% w/w Fuller's Earth and then mixed slowly for 45 minutes at 37 degrees C. After mixing, this mixture was passed through a peristaltic pump and centrifuged, prior to filtration of the liquid through a 10 micron filter, a 2 micron filter, a 0.5 micron filter, and then a 0.2 micron filter. The resulting liquid fraction is the oil base.

This method, along with the method of Example 2, allows production of ZESTRA® without the unappealing smell that consumers can find unappealing, as explained in Example 3, and also produces a stable product with little color.

### Example 2. Preparation of ZESTRA from Oil Base for Topical Application

The oil base was mixed with 0.45889% Coleus forskohli, 0.45889% ascorbyl palmitate, 0.38301% vitamin E (DL-alpha tocopherol), 0.45889% ferulic acid, 0.47696% natural α-Angelica lactone, 0.45889% theobromine, and 0.93947% osthole (35%) and mixed vigorously at 1400 rpms for a minimum of 12 hours at room temperature. After mixing, this mixture was filtered through a 10 micron filter, a 2 micron filter, a 0.5 micron filter, and then a 0.2 micron filter.

The filtered mixture was then combined with peach flavoring (to 1.01%), Magna Sweet® (an ammonium salt form of glycyrrhizic acid) (to 0.5%), and neotame (to 0.25%) and mixed for one hour. The resulting mixture was then filtered through a 0.5 micron filter and then a 0.2 micron filter. The filtrate was ZESTRA®. The ZESTRA® was then bottled in a fiber drum, which was sealed with a nitrogen head cap.

This method allows production of ZESTRA® without the unappealing smell that consumers can find unappealing, as explained in Example 3, and also produces a stable product with little color.

### Example 3. Consumer Survey Demonstrating Effectiveness of ZESTRA®

A consumer survey was conducted with 246 participants to assess the effects of ZESTRA® on sexual arousal, orgasm, sex life satisfaction, smell, ease of use, and consumer behavior. The survey consisted of the following questions and possible answers:

1. Did you find ZESTRA® easy or difficult to use?

—Very difficult —Difficult —Neutral —Easy —Very easy

2. Did you find the instructions easy or difficult to follow?

—Difficult —Easy —Neutral —Very easy

3. Using ZESTRA® were sensations any more intense?

—Much less intense —Less intense —No difference —More intense —Much more intense

4. Using ZESTRA® did you find any difference in how easy it was for you to orgasm?

—Much harder —Less easy —No difference —Easier —Much easier

5. When using ZESTRA® did you find any difference in the intensity of your orgasms?

—Much less intense —Less intense —No difference —More intense —Much more intense

6. When using ZESTRA® did you notice any difference in the frequency of your orgasms?

—Much less frequent —Less frequent —No difference —More frequent —Much more frequent

7. When using ZESTRA® did you notice any difference in your level of arousal?

—Much less aroused —Less aroused —No difference —More aroused —Much more aroused

8. Before using ZESTRA® how would you rate your Overall Satisfaction with your sexual experience?

—Very unsatisfactory —Unsatisfactory —Neutral —Satisfactory —Very satisfactory

9. When using ZESTRA® how would you rate your Overall Satisfaction with your sexual experience?

—Very unsatisfactory —Unsatisfactory —Neutral —Satisfactory —Very satisfactory

10. About how many times do you use ZESTRA® ?

→Twice a week —Twice a week —Once a week —Once a fortnight —Once a month —<once a month

11. How likely are you to continue using ZESTRA® ?

—Very unlikely —Unlikely —Neutral —Likely —Very likely

12. How likely are you to recommend ZESTRA® to others?

—Very unlikely —Unlikely —Neutral —Likely —Very likely

13. How appealing to you find the current ZESTRA® smell?

—Very unappealing —Unappealing —Neutral —Appealing —Very appealing

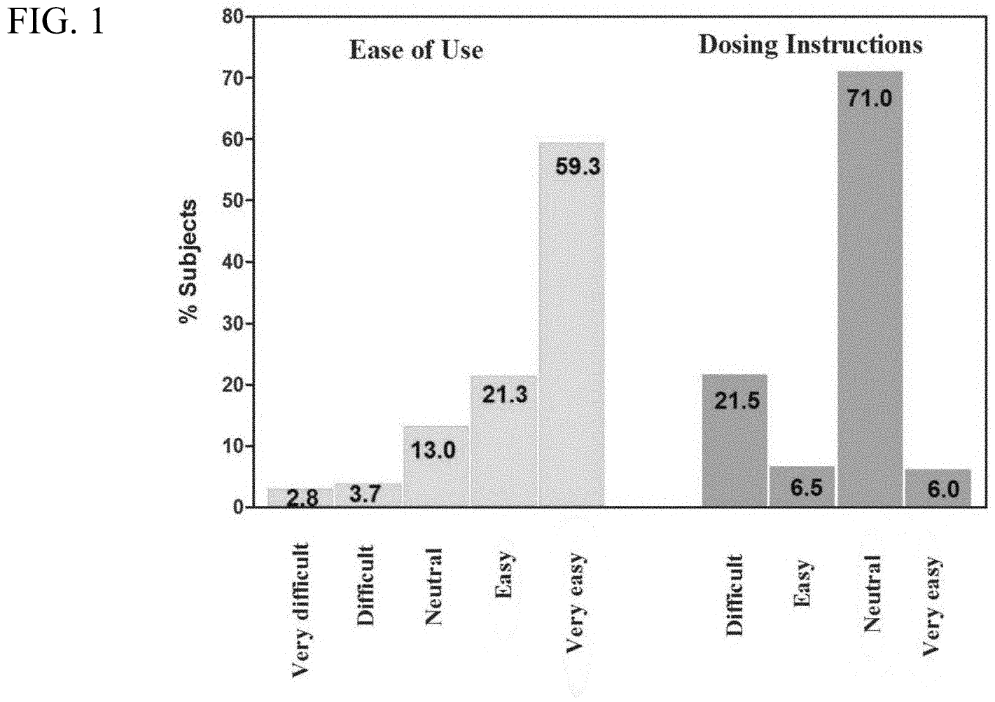

The results demonstrate that consumer found ZESTRA® to be very easy to use (FIG. 1). Most participants had no difficulty understanding or following the dosing instructions (FIG. 1).

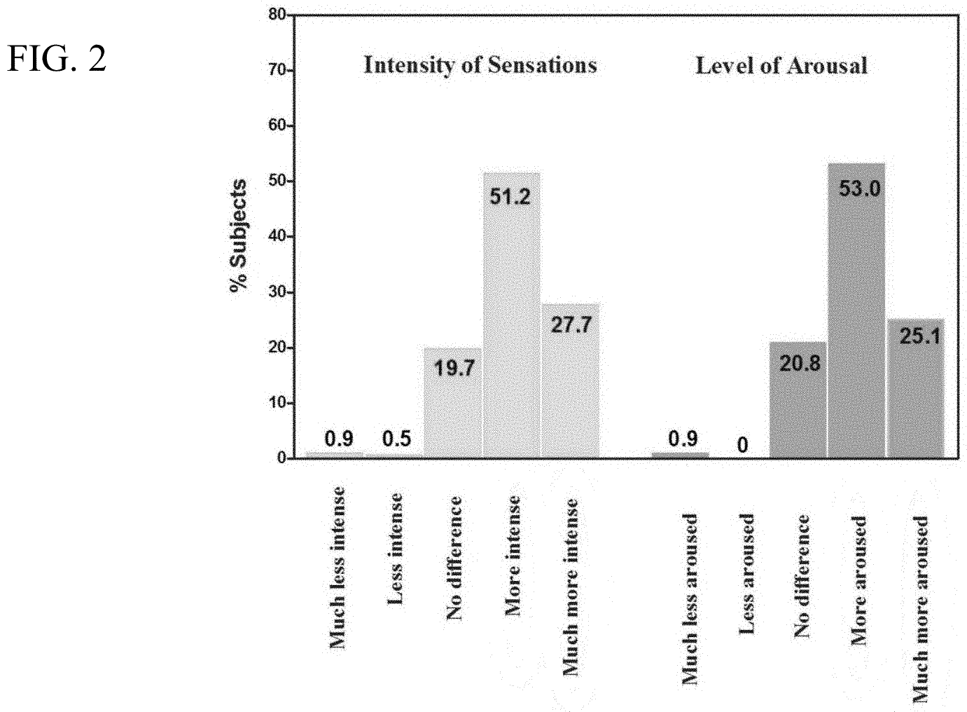

Approximately 78% of participants achieved intense sensations and increased arousal when using ZESTRA°, reporting more or much more intense sensations and more or much more arousal (FIG. 2).

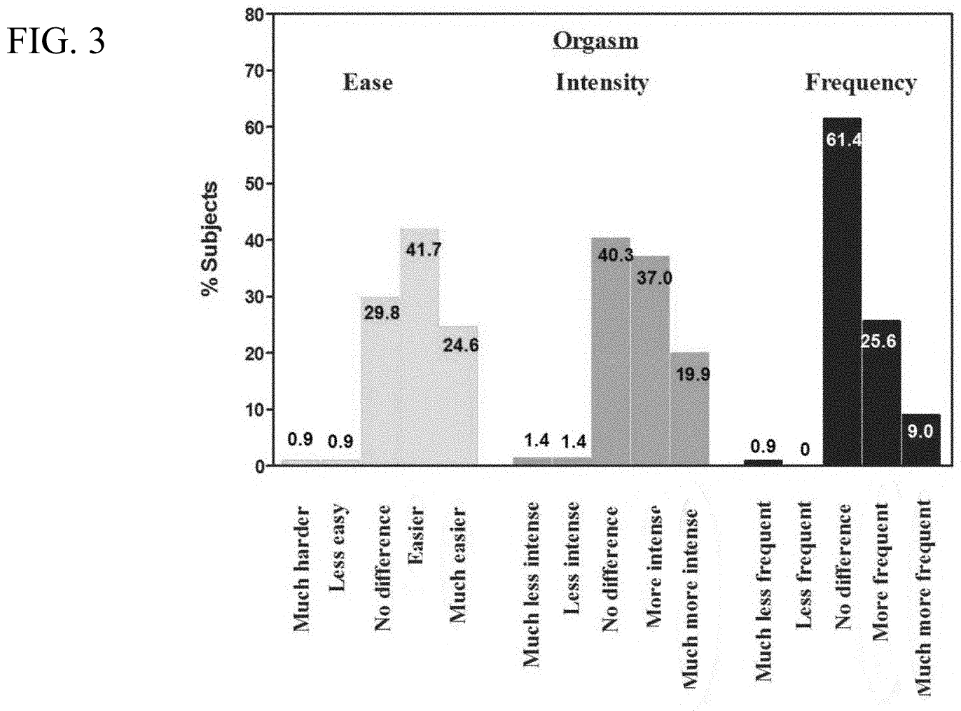

66.3% of participants reported easier or much easier orgasms when using ZESTRA® (FIG. 3). 56.9% of participants reported more intense or much more intense orgasms when using ZESTRA® (FIG. 3). 34.6% or participants reported more or much frequent orgasms when using ZESTRA® (FIG. 3).

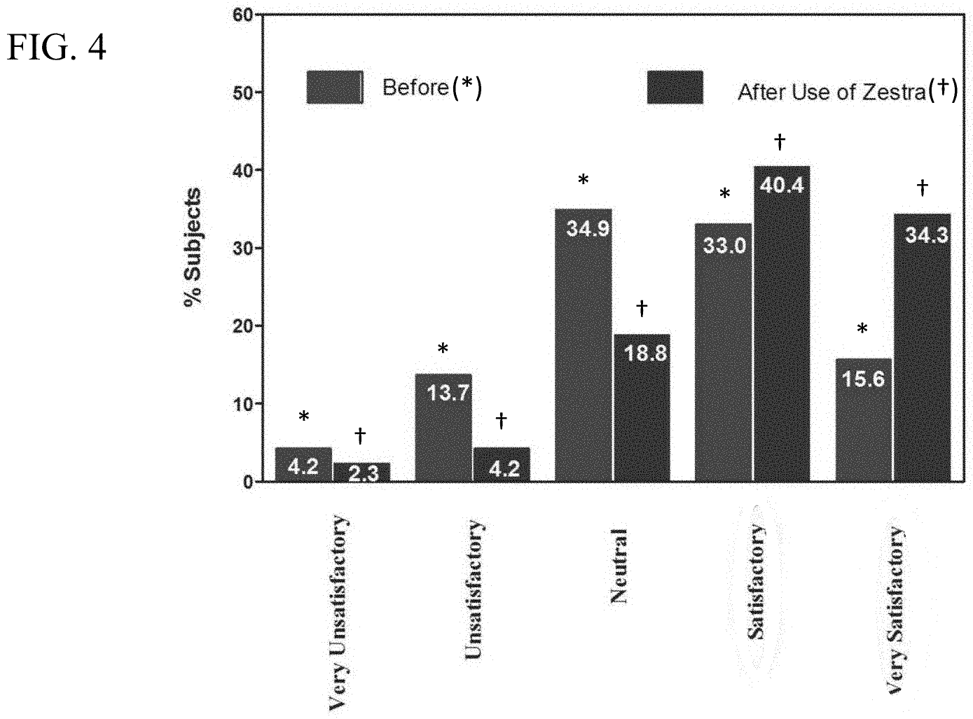

Overall satisfaction improved with sexual experience improved from when using ZESTRA®, with 74.7% of participants reporting satisfactory or very satisfactory overall satisfaction with sexual experience when using ZESTRA® (FIG. 4).

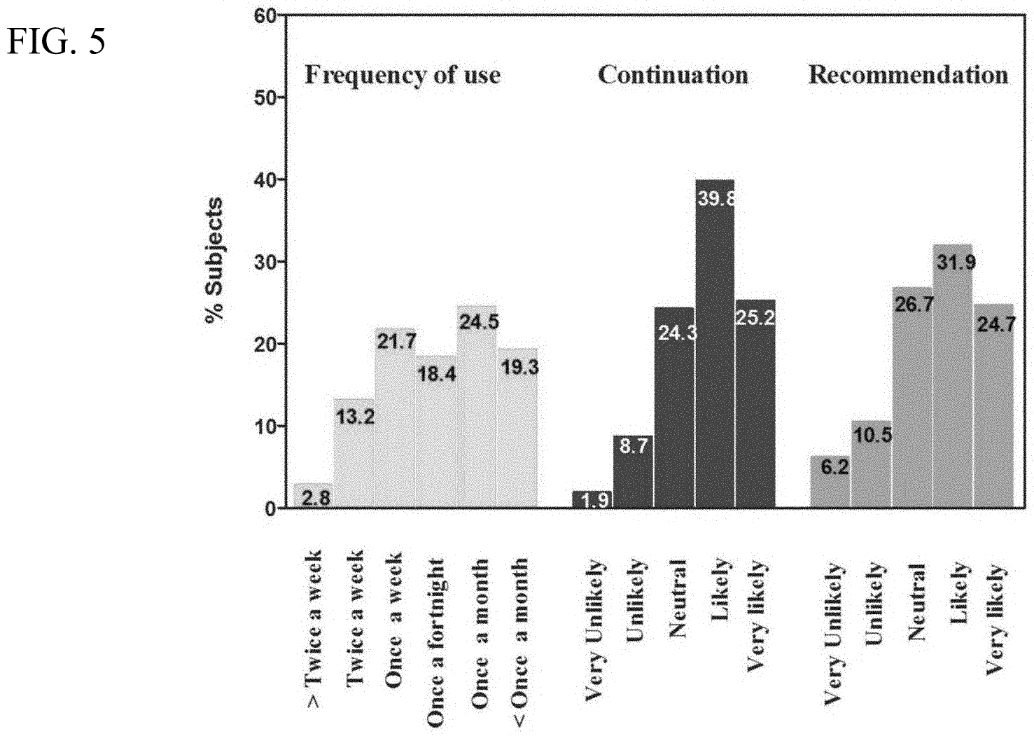

Consumers used ZESTRA® frequently and would continue using it and recommend it to others (FIG. 5). Approximately 80% reported using ZESTRA® at least once a month, approximately 38% at least once a week, and 16% more than once a week. 65% would continue using ZESTRA®, and approximately 57% would recommend it to others.

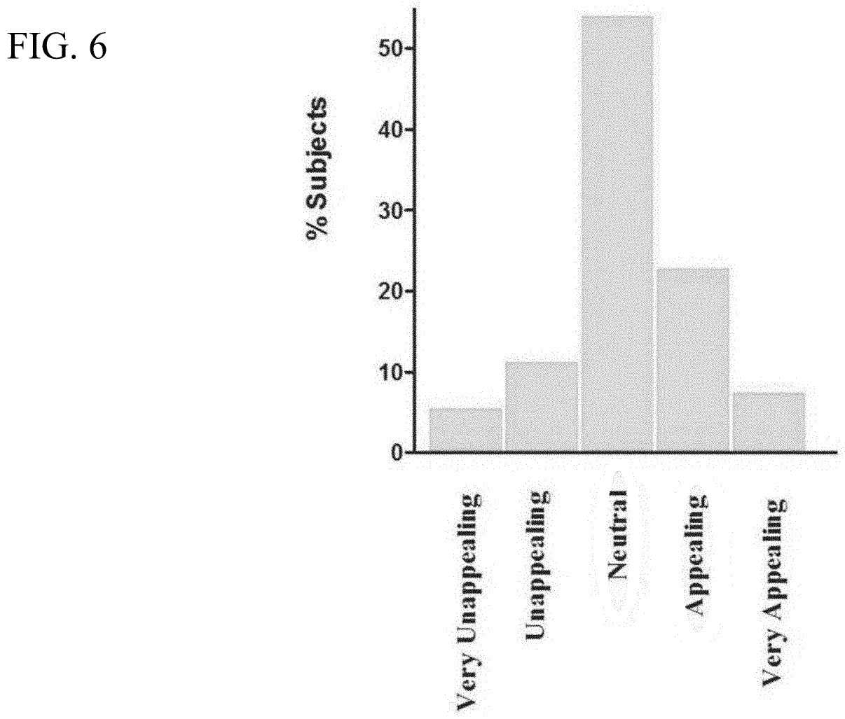

Over 80% reported the smell of ZESTRA® was not unappealing, and approximately 30% even reported the smell to be appealing or very appealing (FIG. 6).

As used herein, the term “about” has its generally accepted meaning. In one embodiment, the term about means±10% of the associated value. For example, about 100 mg means 100 mg±10 mg. In one embodiment, the term about means±5% of the associated value. For example, about 100 mg means 100 mg±5 mg. In one embodiment, the term about means±2% of the associated value. For example, about 100 mg means 100 mg±2 mg. In one embodiment, the term about means±1% of the associated value. For example, about 100 mg means 100 mg±1 mg.

While various embodiments of the present disclosure have been described in detail, it is apparent that modifications and adaptations of those embodiments will occur to those skilled in the art. It is to be expressly understood, however, that such modifications and adaptations are within the scope of the present disclosure, as set forth in the following exemplary claims.

1-11. (canceled)

12. A composition, comprising an oil base of borage seed oil, evening primrose oil, and Angelica pubescens, wherein the oil base is produced by a process comprising:
mixing borage seed oil with evening primrose oil;
mixing Angelica pubescens root with the borage seed oil and evening primrose oil;
clarifying the resulting mixture;
adding Fuller's Earth to the mixture and mixing further to produce a second mixture; and
clarifying the second mixture.

13. The composition of claim 12, wherein the composition does not produce an unappealing odor.

14. The composition of claim 12, further comprising an additional ingredient selected from the group consisting of:
Coleus forskohlii extract;
ascorbyl palmitate;
alpha tocopherol;
ferulic acid;
α-Angelica lactone;
theobromine; and
osthole.

15. The composition of claim 12, wherein the additional ingredient is added to the oil base.

16. The composition of claim 12, wherein the composition does not produce an unappealing odor.

17. The composition of claim 12, further comprising the additional ingredients:
Coleus forskohlii extract;
ascorbyl palmitate;
alpha tocopherol;
ferulic acid;
α-Angelica lactone;
theobromine; and
osthole.

18. The composition of claim 15, wherein the additional ingredients are added to the oil base.

19. The composition of claim 16, wherein the composition does not produce an unappealing odor.

20. A composition, comprising:
about 47% borage seed oil w/w;
about 47% evening primrose oil w/w;
about 64% Angelica pubescens root powder w/w;
about 0.46% Coleus forskohlii extract w/w;
about 0.46% ascorbyl palmitate w/w;
about 0.38% alpha tocopherol w/w;
about 0.46% ferulic acid 0.46% w/w;
about 0.48% α-Angelica lactone w/w; and
about 0.94% osthole w/w.

21. A method of improving a sexual characteristic, comprising administering the therapeutic composition of claim 12 and theobromine to a subject in need thereof.

22. The method of claim 21, wherein the therapeutic composition comprises:
about 47% borage seed oil w/w;
about 47% evening primrose oil w/w; and
about 64% Angelica pubescens root powder w/w.

23. The method of claim 21, wherein therapeutic composition comprises about 0.46% theobromine.

24. The method of claim 21, wherein the therapeutic composition further comprises an ingredient selected from the group consisting of:
Coleus forskohlii extract;
ascorbyl palmitate;
alpha tocopherol;
ferulic acid;
α-Angelica lactone; and
osthole.

25. The method of claim 21, wherein the therapeutic composition further comprises:
Coleus forskohlii extract;
ascorbyl palmitate;
alpha tocopherol;
ferulic acid;
α-Angelica lactone; and
osthole.

26. The method of claim 21, wherein the therapeutic composition further comprises an ingredient selected from the group consisting of:
about 0.46% w/w Coleus forskohlii extract;
about 0.46% w/w ascorbyl palmitate;
about 0.38% w/w alpha tocopherol;
about 0.46% w/w ferulic acid;
about 0.48% w/w α-Angelica lactone; and
about 0.94% w/w osthole.

27. The method of claim 21, wherein the therapeutic composition further comprises:
about 0.46% w/w Coleus forskohlii extract;
about 0.46% w/w ascorbyl palmitate;
about 0.38% w/w alpha tocopherol;
about 0.46% w/w ferulic acid;
about 0.48% w/w α-Angelica lactone; and
about 0.94% w/w osthole.

28. The method of claim 21, wherein the therapeutic composition further comprises flavoring.

29. The method of claim 28, wherein the flavoring is selected from the group consisting of raspberry flavoring, peach flavoring, neotame, ammoniated glycyrrhizin, and monoammonium glycyrrhizinate.

30. The method of claim 21, wherein the method increases intensity of sensations, increases level of arousal, increases orgasm ease, increases intensity, increases frequency, increases overall satisfaction with sexual experience.

31. The method of claim 21, wherein the therapeutic composition does not produce an unappealing odor.
